(19) United States
(12) Patent Application Publication
(10) Pub. No.: US 2024/0109821 A1
(43) Pub. Date: Apr. 4, 2024

(54) PROCESS TO PRODUCE PROPYLENE FROM REFINERY DRY GAS

(71) Applicant: UOP LLC, Des Plaines, IL (US)

(72) Inventors: Xi Zhao, Arlington Heights, IL (US); Ling Zhou, Palatine, IL (US); Jan De Ren, Bracknell (GB)

(21) Appl. No.: 18/474,040

(22) Filed: Sep. 25, 2023

Related U.S. Application Data
(60) Provisional application No. 63/412,396, filed on Oct. 1, 2022.

(51) Int. Cl. C07C 4/06

(52) U.S. Cl. CPC C07C 4/06

(57) ABSTRACT

The process converts ethylene in a dilute ethylene (dry gas) stream that may be derived from an FCC product to propylene. The process includes an oligomerization step to convert ethylene in the dilute ethylene stream to heavier olefins, and a second reaction step to convert the heavier olefins to propylene. The oligomerization catalyst may be an amorphous silica-alumina base with a Group VIII and/or VIB metal. The catalyst is resistant to feed impurities such as hydrogen sulfide, carbon oxides, hydrogen and ammonia. At least 50 wt-% of the ethylene in the dilute ethylene stream can be converted to C₄⁺ olefins and recycled to the FCC for cracking to propylene.

### FIELD

The field is a process for converting dilute ethylene in a hydrocarbon stream to propylene. The process comprises oligomerization integrated with an FCC unit.

### BACKGROUND

Catalytic cracking can create a variety of products from larger hydrocarbons. Often, a feed of a heavier hydrocarbon, such as a vacuum gas oil, is provided to a catalytic cracking reactor, such as a fluid catalytic cracking (FCC) reactor. Various products may be produced from such a system, including a gasoline product and/or light product such as propylene and/or ethylene.

In such systems, secondary reactor(s) can be utilized to further convert low value products from the main reactor. Although additional capital costs may be incurred by using secondary reactor(s), its catalyst, operation conditions and feed (recycled low value stream) can be tailored for maximizing high value products, such as light olefins including propylene and/or ethylene, to provide overall benefit.

Light olefin production is vital to the production of sufficient plastics to meet worldwide demand. Propylene is highly valued for conversion to various grades of polypropylene (PP) and for chemicals production.

Dry gas is the common name for the off-gas stream from a fluid catalytic cracking unit that contains all the gases with boiling points lower than ethane. The off-gas stream is compressed to remove as much of the C₃ and C₄ gases as possible. Sulfur is also largely absorbed from the off-gas stream in a scrubber that utilizes an amine absorbent. The remaining stream is known as the FCC dry gas. A typical dry gas stream contains 5 to 50 wt-% ethylene, 10 to 20 wt-% ethane, 0.5 to 20 wt-% hydrogen, 5 to 20 wt-% nitrogen, about 0.1 to about 5.0 wt-% of each carbon monoxide and carbon dioxide and less than 0.01 wt-% hydrogen sulfide and ammonia with the balance being methane.

Currently, the FCC dry gas stream is sent to a burner as fuel gas. An FCC unit that processes 7,949 kiloliters (50,000 barrels) per day will burn about 181,000 kg (200 tons) of dry gas with about 36,000 kg (40 tons) of ethylene as fuel per day when operating to maximize gasoline production. However, as the FCC operation shifts toward high propylene production, the amount of dry gas produced could reach as high as 427,000 kg (470 tons), with about 210,000 kg (231 tons) of ethylene. Because a large price difference exists between fuel gas and light olefins it would appear economically advantageous to attempt to recover this ethylene. However, the dry gas stream contains impurities that can poison catalysts and is so dilute that ethylene recovery is not economically justified by gas recovery systems.

The oligomerization of olefins to liquid product typically involves the use of propylene or butylene particularly from liquefied petroleum gas (LPG) or dehydrogenated feedstocks to make gasoline or diesel range olefins. Dilute ethylene is little used as an oligomerization feedstock in these processes because of its much lower reactivity. The oligomerization of concentrated ethylene streams to liquid products is not suitable for dry gas ethylene oligomerization because the catalysts are incompatible with the trace impurities. U.S. Pat. No. 8,575,410 describes a process of converting ethylene in dry gas to diesel-range liquid fuel, but this process does not capture the full value of dry gas ethylene because liquid fuel is still less valuable than light olefins.

There is need for further utilization of dilute ethylene in refinery streams.

### BRIEF SUMMARY

We have found that feeding the dry gas stream to an impurity resistant oligomerization catalyst to convert the ethylene to butenes and higher olefins affords a stream for recycle to an FCC unit. We have found most catalysts suitable for oligomerization of ethylene quickly deactivate in the presence of impurities such as carbon oxides, ammonia and hydrogen sulfide. The impurities do not substantially affect a catalyst comprising a metal from Group/s 6, 8, 9 and 10 in the periodic table on amorphous silica-alumina support. Cracking in the FCC of the oligomer stream produces high quantities of propylene from the FCC unit. The unconverted gas can then be burned as fuel gas, but with the more valuable ethylene converted to propylene.

### BRIEF DESCRIPTION OF THE DRAWINGS

The FIGURE is a schematic elevational drawing of a process of the present disclosure.

### Definitions

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

As used herein, the term “predominant” or “predominate” means greater than 50%, suitably greater than 75% and preferably greater than 90%.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “boiling point temperature” means atmospheric equivalent boiling point (AEBP) as calculated from the observed boiling temperature and the distillation pressure, as calculated using the equations furnished in ASTM D1160 appendix A7 entitled “Practice for Converting Observed Vapor Temperatures to Atmospheric Equivalent Temperatures”.

As used herein, the term “True Boiling Point” (TBP) means a test method for determining the boiling point of a material which corresponds to ASTM D-2892 for the production of a liquefied gas, distillate fractions, and residuum of standardized quality on which analytical data can be obtained, and the determination of yields of the above fractions by both mass and volume from which a graph of temperature versus mass % distilled is produced using fifteen theoretical plates in a column with a 5:1 reflux ratio.

As used herein, “pitch” means the hydrocarbon material boiling above about 524° C. (975° F.) AEBP as determined by any standard gas chromatographic simulated distillation method such as ASTM D2887, D6352 or D7169, all of which are used by the petroleum industry.

As used herein, the term “T5” or “T95” means the temperature at which 5 mass percent or 95 mass percent, as the case may be, respectively, of the sample boils using ASTM D-86 or TBP.

As used herein, the term “initial boiling point” (IBP) means the temperature at which the sample begins to boil using ASTM D-7169, ASTM D-86 or TBP, as the case may be.

As used herein, the term “end point” (EP) means the temperature at which the sample has all boiled off using ASTM D-7169, ASTM D-86 or TBP, as the case may be.

As used herein, “vacuum gas oil” means a hydrocarbon material having an IBP of at least about 232° C. (450° F.), a T5 of between about 288° C. (550° F.) and about 392° C. (700° F.), typically no more than about 343° C. (650° F.), a T95 between about 510° C. (950° F.) and about 570° C. (1058° F.) and, or an EP of no more than about 626° C. (1158° F.) prepared by vacuum fractionation of atmospheric residue as determined by any standard gas chromatographic simulated distillation method such as ASTM D2887, D6352 or D7169, all of which are used by the petroleum industry.

As used herein, “atmospheric residue” means a hydrocarbon material having an IBP of at least about 232° C. (450° F.), a T5 of between about 288° C. (550° F.) and about 392° C. (700° F.), typically no more than about 343° C. (650° F.), and a T95 between about 510° C. (950° F.) and about 700° C. (1292° F.) obtained from the bottoms of an atmospheric crude distillation column.

As used herein, “vacuum residuum” means hydrocarbon material boiling with an IBP of at least about 500° C. (932° F.).

### DETAILED DESCRIPTION

The present disclosure is directed to formation of propylene in an FCC unit and may be applied to any hydrocarbon stream containing ethylene and, preferably, a dilute proportion of ethylene. A suitable, dilute ethylene stream may typically comprise between about 5 and about 65 wt-% ethylene. An FCC dry gas stream is a suitable dilute ethylene stream. Other dilute ethylene streams may also be utilized in the present disclosure such as coker dry gas streams. Because the present disclosure is particularly suited to FCC dry gas, the subject application will be described with respect to utilizing ethylene from an FCC dry gas stream.

Now turning to the FIGURE, wherein like numerals designate like components, a process and apparatus generally includes an FCC unit section 6 and a product recovery section 90. The FCC unit section 6 includes a first FCC reactor 10 comprising a first reactor unit 12 and a catalyst regenerator 14. Process conditions in the first FCC reactor 10 may include a cracking reaction temperature of about 400° to about 600° C., preferably about 538° C. to about 593° C. at the reactor outlet, and a catalyst regeneration temperature of about 500° to about 900° C. Both the cracking and regeneration occur at an absolute pressure between about 100 kPa (14 psia) to about 650 kPa (94 psia), preferably between about 140 kPa (20 psia) to about 450 kPa (65 psia).

The FIGURE shows a first FCC reactor vessel 12 in which a first hydrocarbon feedstock in line 15 through a distributor 16 is contacted with a first stream of fluid catalyst entering from a regenerated catalyst standpipe 18 and a recirculation catalyst standpipe 19. The first hydrocarbon feedstock may comprise vacuum gas oil, atmospheric resid, deasphalted oil, vacuum resid or any other stream processed in a conventional FCC unit.

The catalyst can be a single catalyst or a mixture of different catalysts. Usually, the catalyst includes two components or catalysts, namely a first component or catalyst, and a second component or catalyst. Such a catalyst mixture is disclosed in, e.g., U.S. Pat. No. 7,312,370 B2. Generally, the first component may include any of the well-known catalysts that are used in the art of FCC, such as an active amorphous clay-type catalyst and/or a high activity, crystalline molecular sieve. Zeolites may be used as molecular sieves in FCC processes. Preferably, the first component includes a large pore zeolite, such as a Y-type zeolite, an active alumina material, a binder material, including either silica or alumina, and an inert filler such as kaolin.

Typically, the zeolitic molecular sieves appropriate for the first component have a large average pore size. Usually, molecular sieves with a large pore size have pores with openings of greater than about 0.7 nm in effective diameter defined by greater than about 10, and typically about 12, member rings. Pore Size Indices of large pores can be above about 31. Suitable large pore zeolite components may include synthetic zeolites such as X and Y zeolites, mordenite and faujasite. A portion of the first component, such as the zeolite, can have any suitable amount of a rare earth metal or rare earth metal oxide.

The second component may include a medium or smaller pore zeolite catalyst, such as an MFI zeolite, as exemplified by at least one of ZSM-5, ZSM-11, ZSM-12, ZSM-23, ZSM-35, ZSM-38, ZSM-48, and other similar materials. Other suitable medium or smaller pore zeolites include ferrierite, and erionite. Preferably, the second component is a medium or small pore zeolite dispersed on a matrix including a binder material such as silica or alumina and an inert filler material such as kaolin. The second component may also include some other active material such as Beta zeolite. These compositions may have a crystalline zeolite content of about 10 to about 50 wt % or more, and a matrix material content of about 50 to about 90 wt %. Components containing about 40 wt % crystalline zeolite material are preferred, and those with greater crystalline zeolite content may be used. Generally, medium and smaller pore zeolites are characterized by having an effective pore opening diameter of less than or equal to about 0.7 nm, rings of about 10 or fewer members, and a Pore Size Index of less than about 31.

The total catalyst mixture in the first FCC reactor 12 may contain about 1 to about 25 wt % of the second component, namely a medium to small pore crystalline zeolite with greater than or equal to about 1.75 wt % of the second component being preferred. The first component may comprise the balance of the catalyst composition. In some preferred embodiments, the relative proportions of the first and second components in the mixture may not substantially vary throughout the first FCC reactor 12. The high concentration of the medium or small pore zeolite as the second component of the catalyst mixture can improve selectivity to light olefins. In one exemplary embodiment, the second component can be a ZSM-5 zeolite and the mixture can include about 4 to about 10 wt % ZSM-5 zeolite excluding any other components, such as binder and/or filler.

Preferably, at least one of the first and/or second catalysts is an MFI zeolite having a silicon to aluminum ratio greater than about 15, preferably greater than about 75. In one exemplary embodiment, the silicon to aluminum ratio can be about 15:1 to about 35:1.

The contacting may occur in a narrow first riser 20, extending upwardly to the bottom of a first reactor vessel 22. The contacting of the first hydrocarbon feedstock and the first stream of fluid catalyst is fluidized by gas such as steam from a fluidizing distributor 24. In an embodiment, heat from the catalyst vaporizes the first hydrocarbon feedstock, and the first hydrocarbon feedstock is thereafter cracked to a first cracked product stream of lighter molecular weight in the presence of the first catalyst stream as both are transferred up the riser 20 into the reactor vessel 22 providing a first mixture of catalyst and product gases.

The pressure in the first riser 20 may be about 200 kPa (29 psia) to about 450 kPa (65 psia), but it could be lower. A steam rate of about 3 to about 7 wt % of the first hydrocarbon feedstock is added to the first riser 20. Inevitable side reactions occur in the first riser 20 leaving coke deposits on the catalyst that lower catalyst activity to provide a spent catalyst stream. The first cracked product stream in the first mixture of catalyst and product gases is thereafter separated from the spent catalyst stream using cyclonic separators which may include one or two stages of cyclones 62 in the reactor vessel 22. A gaseous, first cracked product stream exits the reactor vessel 22 through a first product outlet 31 to line 32 for transport to the downstream product recovery section 90.

The spent or coked catalyst requires regeneration for further use. The spent catalyst stream, after separation from the first cracked product stream by means of a disengagement device 54 in a first disengagement chamber 56, falls into a stripping section 34 where steam is injected through a distributor 35 to purge any residual hydrocarbon vapor. A transport conduit 60 carries the hydrocarbon vapors, including stripped hydrocarbons, stripping media and entrained catalyst to one or two stages of cyclones 62 in the first reactor vessel 22 which separates coked catalyst from the hydrocarbon vapor stream. After the stripping operation, the stripped coked catalyst is carried to the catalyst regenerator 14 through a spent catalyst standpipe 36. Another portion of the stripped coked catalyst may be recycled to the riser 20 by the recirculation catalyst standpipe 19 without undergoing regeneration.

The FIGURE depicts a regenerator 14 known as a combustor. However, other types of regenerators are suitable. In the catalyst regenerator 14, a stream of oxygen-containing gas, such as air, is introduced through an air distributor 38 to contact the coked catalyst. Coke is combusted from the coked catalyst in a combustion chamber 80 to provide regenerated catalyst and flue gas. The catalyst regeneration process adds a substantial amount of heat to the catalyst, providing energy to offset the endothermic cracking reactions occurring in the reactor riser 20. Catalyst and air flow upwardly together in the combustion chamber 80 of regenerator 14 and, after regeneration, are initially separated by discharge through a disengager 40 and enter a separation chamber 86. Additional recovery of the regenerated catalyst and flue gas exiting the disengager 40 is achieved using first and second stage separator cyclones 44, 46, respectively within the separation chamber 86 of the catalyst regenerator 14. Catalyst separated from flue gas dispenses through diplegs from cyclones 44, 46 while flue gas relatively lighter in catalyst sequentially exits cyclones 44, 46 and exits the regenerator vessel 14 through flue gas outlet 47 in flue gas line 48. Regenerated catalyst is carried back to the riser 20 through the regenerated catalyst standpipe 18. As a result of the coke burning, the flue gas vapors exiting at the top of the catalyst regenerator 14 in line 48 contain CO, CO₂, N₂ and H₂O, along with smaller amounts of other species. Hot flue gas exits the regenerator 14 through the flue gas outlet 47 in a line 48 for further processing.

The product recovery section 90 is in downstream communication with the product outlet 31 and second product outlet 231. “Communication” means that material flow is operatively permitted between enumerated components. In the product recovery section 90, the gaseous FCC product in line 32 is directed via a combined gaseous FCC product in line 33 to a lower section of an FCC main fractionation column 92. The main column 92 is in downstream communication with the product outlet 31 and secondary product outlet 231. Several fractions of FCC product may be separated and taken from the main column including a heavy slurry oil from the bottoms in line 93, a heavy cycle oil stream in line 94, a light cycle oil in line 95 taken from outlet 95a and a heavy naphtha stream in line 96 taken from outlet 96a. Any or all of lines 93-96 may be cooled and pumped back to the main column 92 to cool the main column typically at a higher location. Gasoline and gaseous light hydrocarbons are removed in overhead line 97 from the main column 92 and condensed before entering a main column receiver 99. The main column receiver 99 is in downstream communication with the product outlet 31 and secondary product outlet 231, and the main column 92 is in upstream communication with the main column receiver 99. “Upstream communication” means that at least a portion of the material flowing from the component in upstream communication may operatively flow to the component with which it communicates.

An aqueous stream is removed from a boot in the receiver 99. Moreover, a condensed light naphtha stream is removed in line 101 while an overhead stream is removed in line 102. The overhead stream in line 102 contains gaseous light hydrocarbon which may comprise a dilute ethylene stream. The streams in lines 101 and 102 may enter a vapor recovery section 120 of the product recovery section 90.

The vapor recovery section 120 is shown to be an absorption-based system, but any vapor recovery system may be used including a cold box system. To obtain sufficient separation of light gas components the gaseous stream in line 102 is compressed in compressor 104. More than one compressor stage may be used, but typically a dual stage compression is utilized. The compressed light hydrocarbon stream in line 106 is joined by streams in lines 107 and 108, chilled and delivered to a high-pressure receiver 110. An aqueous stream in line 111 from the receiver 110 may be routed to the main column receiver 99. A gaseous hydrocarbon stream in line 112 comprising the dilute ethylene stream is routed to a primary absorber 114 in which it is contacted with unstabilized gasoline from the main column receiver 99 in line 101 to effect a separation between C₃⁺ and C₂⁻ hydrocarbons. The primary absorber 114 is in downstream communication with the main column receiver 99. A liquid C₃⁺ stream in line 107 is returned to line 106 prior to chilling. A primary off-gas stream in line 116 from the primary absorber 114 comprises the dilute ethylene stream for purposes of the present disclosure. However, to concentrate the ethylene stream further and to recover heavier components line 116 may optionally be directed to a secondary absorber 118, where a circulating stream of light cycle oil in line 121 diverted from line 95 absorbs most of the remaining C₅⁺ and some C₃-C₄ material in the primary off-gas stream. The secondary absorber 118 is in downstream communication with the primary absorber 114. Light cycle oil from the bottom of the secondary absorber in line 119 richer in C₃⁺ material is returned to the main column 92 via the pump-around for line 95. The overhead of the secondary absorber 118 comprising dry gas of predominantly C₂⁻ hydrocarbons with hydrogen sulfide, ammonia, carbon oxides and hydrogen is removed in a secondary off-gas stream in line 122 to comprise a dilute ethylene stream.

Liquid from the high-pressure receiver 110 in line 124 is sent to a stripper 126. Most of the C₂⁻ is removed in the overhead of the stripper 126 and returned to line 106 via overhead line 108. A liquid bottoms stream from the stripper 126 is sent to a first debutanizer column 130 in a bottoms line 128. The first debutanizer column 130 provides an overhead stream in line 132 comprising a C₃-C₄ hydrocarbon stream from the first debutanizer column. A bottoms stream in line 134 may comprise a first debutanized naphtha stream.

In a first embodiment, a first recycle light cracked naphtha stream may be taken in line 137 through a control valve thereon from the first debutanized naphtha stream in line 134 while the remainder of the first debutanized naphtha stream in line 141 may be further processed into gasoline or other products through a control valve thereon. In this embodiment it is envisioned that a naphtha splitter column 136 may be located upstream in the product recovery section 90.

In an alternative embodiment, the first debutanized naphtha stream in line 134 may be fed to the naphtha splitter column 136 in line 142 through a control valve thereon. In this alternative embodiment, the naphtha splitter column is located downstream in the product recovery section 90 as depicted in the FIGURE and the control valves on lines 137 and 141 will be closed or primarily closed. The naphtha splitter column separates the debutanized naphtha stream into a first split light naphtha stream in an overhead line 138 comprising C₅-C₇ hydrocarbons and a heavy naphtha stream in a bottoms line 140. An alternative first recycle light cracked naphtha stream may be taken in line 139 through a control valve thereon while the remainder of the first split light naphtha stream in the overhead line 138 may be further processed into gasoline or other products.

The C₃-C₄ hydrocarbon stream taken in line 132 may be separated in a C₃-C₄ splitter column 144 into a C₃ hydrocarbon stream in an overhead line 146 and a C₄ hydrocarbon stream in a bottoms line 148. A first recycle C₄ hydrocarbon stream may be taken in line 149 through a control valve thereon while a second C₄ hydrocarbon stream may be further processed into other products in line 147. The C₃ hydrocarbon stream in the overhead line 146 may be further processed for propylene recovery.

One or both of the first recycle light cracked naphtha stream taken in line 137 from the first debutanized naphtha stream in line 134 from the first debutanizer column 130 in downstream communication with the main column 92 comprising olefinic C₅-C₇ hydrocarbons or the alternative first recycle light cracked naphtha stream taken from an overhead line 138 of the naphtha splitter column 136 in downstream communication with the main column 92 in line 139 comprising olefinic C₅-C₇ hydrocarbons and the first recycle C₄ stream comprising olefinic C₄ hydrocarbons taken from a bottoms line 148 of the C₃-C₄ splitter column also in downstream communication with the main column 92 in line 149 may be recycled to a second FCC reactor 202 in a second charge line 150 as the second hydrocarbon stream. The second hydrocarbon stream may be preheated to a temperature of about 221° C. (400° F.) to about 704° C. (1300° F.) and charged to the second FCC reactor 202.

The dilute ethylene stream may be the secondary off-gas stream in line 122 may comprise an FCC dry gas stream comprising between about 5 and about 65 wt-% ethylene and preferably about 10 to about 55 wt-% ethylene. Methane will typically be the predominant component in the dilute ethylene stream at a concentration of between about 25 and about 55 wt-% with ethane being substantially present at typically between about 5 and about 45 wt-%. Between about 0.5 and about 25 wt-% and typically about 1 to about 20 wt-% of hydrogen and nitrogen each may be present in the dilute ethylene stream. Saturation levels of water may also be present in the dilute ethylene stream. If secondary absorber 118 is used, no more than about 5 wt-% of C₃⁺ will be present with typically less than 0.5 wt-% propylene.

Besides hydrogen, other impurities such as hydrogen sulfide, ammonia, carbon oxides and acetylene may also be present in the dilute ethylene stream.

We have found that many impurities in a dry gas ethylene stream can poison an oligomerization catalyst. Hydrogen and carbon monoxide can reduce the metal sites to inactivity. Carbon dioxide and ammonia can attack acid sites on the catalyst. Hydrogen sulfide can attack metals on a catalyst to produce metal sulfides. Acetylene can polymerize and gum up on the catalyst or equipment.

The secondary off-gas stream in line 122, comprising a dilute ethylene stream may be introduced into an optional amine absorber unit 160 to remove hydrogen sulfide to lower concentrations. A lean aqueous amine solution, such as comprising monoethanol amine or diethanol amine, is introduced via line 162 into absorber 160 and is contacted with the flowing secondary off-gas stream to absorb hydrogen sulfide, and a rich aqueous amine absorption solution containing hydrogen sulfide is removed from absorption zone 160 via line 163 and recovered and perhaps further processed.

The amine-treated dilute ethylene stream in line 164 may be introduced into an optional water wash unit 166 to remove residual amine carried over from the amine absorber 160 and reduce the concentration of ammonia and carbon dioxide in the dilute ethylene stream in line 164. Water is introduced to the water wash in line 165. The water in line 165 is typically slightly acidified to enhance capture of basic molecules such as the amine. An aqueous stream in line 167 rich in amine and potentially ammonia and carbon dioxide leaves the water wash unit 166 and may be further processed.

In an aspect, the optionally amine treated dilute ethylene and perhaps water washed stream in line 168 may then be treated in an optional guard bed 170 to remove one or more of the impurities such as carbon monoxide, hydrogen sulfide and ammonia down to lower concentrations. The guard bed 170 may contain an adsorbent to adsorb impurities such as hydrogen sulfide that may poison an oligomerization catalyst. The guard bed 170 may contain multiple adsorbents for adsorbing more than one type of impurity. A typical adsorbent for adsorbing hydrogen sulfide is ADS-12, for adsorbing CO is ADS-106 and for adsorbing ammonia is UOP MOLSIV 3A, all available from UOP, LLC. The adsorbents may be mixed in a single bed or can be arranged in successive beds.

In another aspect, the optionally amine treated dilute ethylene and perhaps water washed stream in line 168 may be treated to recover or reduce hydrogen concentration in the dilute ethylene stream in line 168 to a lower level. In an exemplary embodiment, unit 170 is a pressure swing adsorption (PSA) unit instead of a guard bed. The optionally amine treated dilute ethylene and perhaps water washed stream in line 168 may be passed to a pressure swing adsorption (PSA) unit 170 to recover or reduce hydrogen concentration in the dilute ethylene stream in line 168 to a lower level. The optionally amine treated dilute ethylene and perhaps water washed stream in line 168 may enter the PSA 170 at a high pressure. The molecules heavier than hydrogen present in the optionally amine treated dilute ethylene and perhaps water washed stream in line 168 are adsorbed on the adsorbent while hydrogen molecules do not adsorb on the adsorbent and pass through the bed. The adsorbent may be selected from one or more of silica gel, alumina, activated carbon, molecular sieve including but not limited to zeolite, Metal Organic Framework (MOFs), etc. or their mixture. The PSA 170 may comprise a single layer or multi-layer bed of adsorbent of different composition. The light molecules such as hydrogen leave the PSA 170 at high pressure in line 173 and are recovered. The adsorbed heavy molecules are then desorbed and recovered at a lower pressure from the PSA 170 in a PSA tail gas stream comprising ethylene in line 171.

In an embodiment, the PSA unit 170 may be operated at an adsorption pressure of about 1500 kPa (220 psi) to 3500 kPa (515 psi), and a desorption pressure of about 100 kPa (15 psi) to about 300 kPa (44 psi).

In another aspect, the optionally amine treated dilute ethylene and perhaps water washed stream in line 168 may be passed through a membrane unit 170 to recover or reduce hydrogen concentration in the dilute ethylene stream in line 168 to a lower level. In the membrane unit 170, the optionally amine treated dilute ethylene and perhaps water washed stream in line 168 is passed through a membrane at a high pressure. In an exemplary embodiment, the membrane unit 170 may comprise a polyimide or a polyethersulfone-polyimide blend membrane. Other suitable membranes may include a polymer, porous ceramic, dense ceramic, or metallic membranes. After contact, the molecules heavier than hydrogen present in the optionally amine treated dilute ethylene and perhaps water washed stream in line 168 are mostly rejected by membrane as retentate. The heavy molecules comprising ethylene leave the membrane unit 170 at a high pressure and withdrawn in a retentate stream comprising ethylene in line 171 from the membrane unit 170. Hydrogen present in the optionally amine treated dilute ethylene and perhaps water washed stream in line 168 permeates through the membrane at higher selectivity than the retained molecules and the hydrogen is recovered at the other side of membrane at a lower pressure in a hydrogen stream in line 173.

The membrane unit 170 may be operated at a temperature of about 40° C. (104° F.) to about 80° C. (176° F.). The differential pressure across the membrane can be as low as about 70 kPa (10 psi) or as high as 14.5 MPa (2100 psi) depending on many factors such as the particular membrane used, the flow rate of the inlet stream and the availability of a compressor to compress the permeate stream if such compression is desired. In an embodiment, the differential pressure across the membrane may range between about 446 kPa (50 psig) and about 6996 kPa (1000 psig) feed pressure.

A dilute ethylene stream in line 171 perhaps amine treated, perhaps water washed and perhaps adsorption treated to remove more hydrogen, hydrogen sulfide, ammonia and carbon monoxide will typically have at least one of the following impurity concentrations: about 0.1 wt-% and up to about 5.0 wt-% of carbon monoxide and/or about 0.1 wt-% and up to about 5.0 wt-% of carbon dioxide, and/or at least about 1 wppm and up to about 500 wppm hydrogen sulfide and/or at least about 1 and up to about 500 wppm ammonia, and/or at least about 0.1 and up to about 10 wt-% hydrogen. The type of impurities present and their concentrations will vary depending on the processing and origin of the dilute ethylene stream.

Line 171 carries the dilute ethylene stream to a compressor 172 to be pressured up to reactor pressure. The compressor 172 is in downstream communication with the main column 92, the product recovery section 90, the first product outlet 31 and the second product outlet 231. The compressed dilute ethylene stream can be compressed to at least about 3,550 kPa (500 psia) and perhaps no more than about 10,445 kPa (1500 psia) and suitably between about 4,930 kPa (700 psia) and about 7,687 kPa (1100 psia). It is preferred that the dilute ethylene stream be pressured up to above the critical pressure of ethylene which is about 4,992 kPa (724 psia) for pure ethylene to avoid rapid catalyst deactivation. The compressor 172 may comprise one or more stages with interstage cooling. A heater may be required to bring the compressed stream up to reaction temperature. The compressed dilute ethylene is carried in line 174 to oligomerization reactor 176.

The oligomerization reactor 176 is in downstream communication with the compressor 172 and the primary and secondary absorbers 114 and 118, respectively. The oligomerization reactor preferably contains a fixed catalyst bed 178. The dilute ethylene feed stream contacts the catalyst preferably in a down flow operation. However, upflow operation may be suitable. In an embodiment, the second C₄ hydrocarbon stream in line 147 may also contact the catalyst through line 175 through a valve controlled thereon. The catalyst is preferably an amorphous silica-alumina base with a metal from Group 6, 8, 9 and 10 in the periodic table in the periodic table using IUPAC notations. In an aspect, the catalyst has a Group 8, 9, or 10 metal (hereinafter 8-10) promoted with a Group 6 metal. In an aspect, the catalyst may have a silica-to-alumina ratio of no more than 30 and preferably no more than 20. Typically, the silica and alumina will only be in the base, so the silica-to-alumina ratio will be the same for the catalyst as for the base. The metals can either be impregnated onto or ion exchanged with the silica-alumina base. Co-mulling is also contemplated. Additionally, a suitable catalyst will have a surface area of between about 50 and about 400 m²/g as determined by nitrogen BET.

A preferred oligomerization catalyst of the present disclosure is described as follows. The preferred oligomerization catalyst comprises an amorphous silica-alumina support. One of the components of the catalyst support utilized in the present disclosure is alumina. The alumina may be any of the various hydrous aluminum oxides or alumina gels such as alpha-alumina monohydrate of the boehmite or pseudo-boehmite structure, alpha-alumina trihydrate of the gibbsite structure, beta-alumina trihydrate of the bayerite structure, and the like. A particularly preferred alumina is available from Sasol North America Alumina Product Group under the trademark Catapal. This material is an extremely high purity alpha-alumina monohydrate (pseudo-boehmite) which after calcination at a high temperature has been shown to yield a high purity gamma-alumina. Another component of the catalyst support is an amorphous silica-alumina. A suitable silica-alumina with a silica-to-alumina ratio of 2.6 is available from CCIC, a subsidiary of JGC, Japan.

Another component utilized in the preparation of the catalyst utilized in the present disclosure is a surfactant. The surfactant is preferably admixed with the hereinabove described alumina and the silica-alumina powders. The resulting admixture of surfactant, alumina and silica-alumina is then formed, dried and calcined as hereinafter described. The calcination effectively removes by combustion the organic components of the surfactant but only after the surfactant has dutifully performed its function in accordance with the present disclosure. Any suitable surfactant may be utilized in accordance with the present disclosure. A preferred surfactant is a surfactant selected from a series of commercial surfactants sold under the trademark “Antarox” by Solvay S. A. The “Antarox” surfactants are generally characterized as modified linear aliphatic polyethers and are low-foaming biodegradable detergents and wetting agents.

A suitable silica-alumina mixture may be prepared by mixing proportionate volumes of silica-alumina and alumina to achieve the desired silica-to-alumina ratio. In an embodiment, 85 wt-% amorphous silica-alumina with a silica-to-alumina ratio of 2.6 and 15 wt-% alumina powder will provide a suitable support. In an embodiment, ratios other than 85-to-15 of amorphous silica-alumina to alumina may be suitable.

Any convenient method may be used to incorporate a surfactant with the silica-alumina and alumina mixture. The surfactant is preferably admixed during the admixture and formation of the alumina and silica-alumina. A preferred method is to admix an aqueous solution of the surfactant with the blend of alumina and silica-alumina before the final formation of the support. It is preferred that the surfactant be present in the paste or dough in an amount from about 0.01 to about 10 wt-% based on the weight of the alumina and silica-alumina.

Monoprotic acid such as nitric acid or formic acid may be added to the mixture in aqueous solution to peptize the alumina in the binder. Additional water may be added to the mixture to provide sufficient wetness to constitute a dough with sufficient consistency to be extruded or spray dried.

The paste or dough may be prepared in the form of shaped particulates, with the preferred method being to extrude the dough mixture of alumina, silica-alumina, surfactant and water through a die having openings therein of desired size and shape, after which the extruded matter is broken into extrudates of desired length and dried. A further step of calcination may be employed to give added strength to the extrudate. Generally, calcination is conducted in a stream of dry air at a temperature from about 260° C. (500° F.) to about 815° C. (1500° F.).

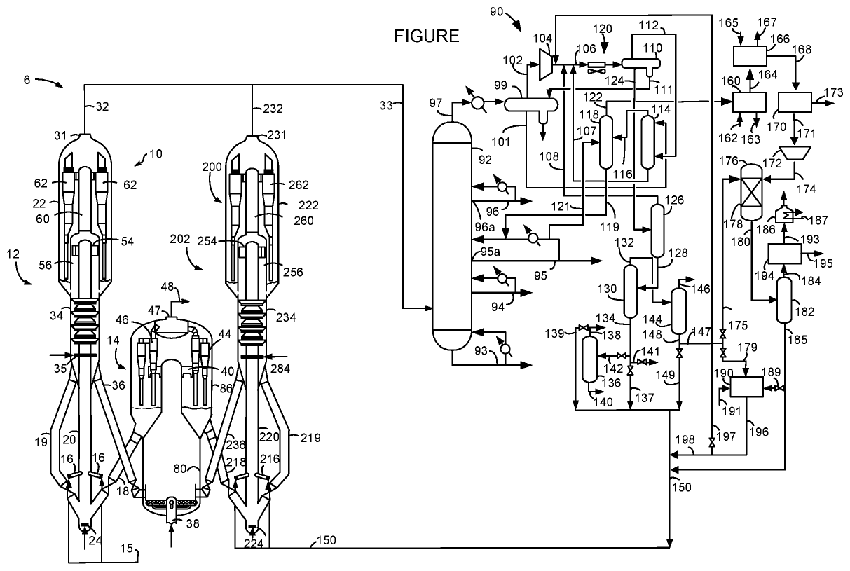

The extruded particles may have any suitable cross-sectional shape, i.e., symmetrical or asymmetrical, but most often have a symmetrical cross-sectional shape, preferably a spherical, cylindrical or polylobal shape. The cross-sectional diameter of the particles may be as small as 40 μm; however, it is usually about 0.635 mm (0.025 inch) to about 12.7 mm (0.5 inch), preferably about 0.79 mm ( 1/32 inch) to about 6.35 mm (0.25 inch), and most preferably about 1.06 mm ( 1/24 inch) to about 4.23 mm (⅙ inch). Among the preferred catalyst configurations are cross-sectional shapes resembling that of a three-leaf clover, as shown, for example, in FIGS. 8 and 8A of U.S. Pat. No. 4,028,227. Preferred clover-shaped particulates are such that each “leaf” of the cross-section is defined by about a 270° arc of a circle having a diameter between about 0.51 mm (0.02 inch) and 1.27 mm (0.05 inch). Other preferred particulates are those having quadralobal cross-sectional shapes, including asymmetrical shapes, and symmetrical shapes such as in FIG. 10 of U.S. Pat. No. 4,028,227.

Typical characteristics of the amorphous silica-alumina supports utilized herein are a total pore volume, average pore diameter and surface area large enough to provide substantial space and area to deposit the active metal components. The total pore volume of the support, as measured by conventional mercury porosimeter methods, is usually about 0.2 to about 2.0 mL/gram, preferably about 0.25 to about 1.0 mL/gram and most preferably about 0.3 to about 0.9 mL/gram. Ordinarily, the amount of pore volume of the support in pores of diameter greater than 100 angstroms is less than about 0.1 mL/gram, preferably less than 0.08 mL/gram, and most preferably less than about 0.05 mL/gram. Surface area, as measured by the B.E.T. method, is typically above 50 m²/gram, e.g., above about 200 m²/gram, preferably at least 250 m²/gram, and most preferably about 300 m²/gram to about 400 m²/gram.

To prepare the catalyst, the support material is compounded, as by a single impregnation or multiple impregnations of a calcined amorphous refractory oxide support particles, with one or more precursors of at least one metal component from Group/s 6, 8, 9 and 10 in the periodic table. The Group 8-10 metal, preferably nickel, should be present in a concentration of about 0.5 to about 15 wt-% and the Group 6 metal, preferably tungsten, should be present in a concentration of about 0 to about 12 wt-%. The impregnation may be accomplished by any method known in the art, as for example, by spray impregnation wherein a solution containing the metal precursors in dissolved form is sprayed onto the support particles. Another method is the multi-dip procedure wherein the support material is repeatedly contacted with the impregnating solution with or without intermittent drying. Yet other methods involve soaking the support in a large volume of the impregnation solution or circulating the support therein, and yet one more method is the pore volume or pore saturation technique wherein support particles are introduced into an impregnation solution of volume just sufficient to fill the pores of the support. On occasion, the pore saturation technique may be modified so as to utilize an impregnation solution having a volume between 10 percent less and 10 percent more than that which will just fill the pores.

The catalyst may be further modified by compounding one or more precursors of metal component from Groups 1 and 2 in the periodic table, such as sodium, onto the support particles. The Groups 1 and 2 metal precursors may be introduced by impregnation following any method known in the art, as described previously, and it may be either together or separately from the impregnation of Groups 6, 8, 9 and 10 metal precursors.

If the active metal precursors are incorporated by impregnation, a subsequent or second calcination at elevated temperatures, as for example, between 399° and 760° C. (750° and 1400° F.), converts the metals to their respective oxide forms. In some cases, calcinations may follow each impregnation of individual active metals. A subsequent calcination yields a catalyst containing the active metals in their respective oxide forms.

A preferred oligomerization catalyst of the present disclosure has an amorphous silica-alumina base impregnated with 0.5-15 wt-% nickel in the form of 3.175 mm (0.125 inch) extrudates and a density of about 0.45 to about 0.65 g/mL. It is also contemplated that metals can be incorporated onto the support by other methods such as ion-exchange and co-mulling.

An alternative catalyst suitable for the present disclosure utilizes a co-gelled silica-alumina support made by the well-known oil-drop method which permits the utilization of the support in the form of macrospheres. For example, an alumina sol, utilized as an alumina source, is commingled with an acidified water glass solution as a silica source, and the mixture is further commingled with a suitable gelling agent, for example, urea, hexamethylenetetramine, or mixtures thereof. The mixture is discharged while still below gellation temperature, and by means of a nozzle or rotating disk, into a hot oil bath maintained at gellation temperature. The mixture is dispersed into the oil bath as droplets which form into spheroidal gel particles during passage therethrough. The alumina sol is preferably prepared by a method wherein aluminum pellets are commingled with a quantity of treated or deionized water, with hydrochloric acid being added thereto in a sufficient amount to digest a portion of the aluminum metal and form the desired sol. A suitable reaction rate is affected at about reflux temperature of the mixture.

The spheroidal gel particles prepared by the oil-drop method are aged, usually in the oil bath, for a period of at least 10 to 16 hours, and then in a suitable alkaline or basic medium for at least 3 to about 10 hours, and finally water-washed. Proper gellation of the mixture in the oil bath, as well as subsequent aging of the gel spheres, is not readily accomplished below about 48.9° C. (120° F.), and at about 98.9° C. (210° F.), the rapid evolution of the gases tends to rupture and otherwise weaken the spheres. By maintaining sufficient superatmospheric pressure during the forming and aging steps in order to maintain water in the liquid phase, a higher temperature can be employed, frequently with improved results. If the gel particles are aged at superatmospheric pressure, no alkaline aging step is required.

The spheres are water-washed, preferably with water containing a small amount of ammonium hydroxide and/or ammonium nitrate. After washing, the spheres are dried, at a temperature of from about 93.3° C. (200° F.) to about 315° C. (600° F.) for a period of from about 6 to about 24 hours or more, and then calcined at a temperature of from about 426.67° C. (800° F.) to about 760° C. (1400° F.) for a period of from 2 to about 12 hours or more.

The Group 8-10 component and the Group 6 component, and optionally Group 1 and 2 components, are composed with the co-gelled silica-alumina carrier material by any suitable co-impregnation technique. Thus, the carrier material can be soaked, dipped, suspended, or otherwise immersed in an aqueous impregnating solution containing a soluble Group 8-10 salt and a soluble Group 6 salt. One suitable method comprises immersing the carrier material in the impregnating solution and evaporating the same to dryness in a rotary steam dryer, the concentration of the impregnating solution being such as to ensure a final catalyst composite comprising an atomic ratio of nickel to nickel plus tungsten of about 0.1 to about 0.3. Another suitable method comprises dipping the carrier material into the aqueous impregnating solution at room temperature until complete penetration of carrier by the solution is achieved. After absorption of the impregnating solution, the carrier is drained of free surface liquid and dried in a moving belt calciner. Yet another suitable method comprises adding Group 8-10 component and/or the Group 6 component to the mixture comprising alumina source, silica source and the gelling agent before co-gelling step.

The catalyst composite is usually dried at a temperature of from about 93.3° C. (200° F.) to about 260° C. (500° F.) for a period of from about 1 to about 10 hours prior to calcination. In accordance with the present disclosure, calcination is effected in an oxidizing atmosphere at a temperature of from about 371° C. (700° F.) to about 650° C. (1200° F.). The oxidizing atmosphere is suitably air, although other gases comprising molecular oxygen may be employed.

A suitable alternative catalyst is an oil dropped silica-alumina spherical support with a diameter of 3.175 mm (0.125 inch) impregnated with about 0.5 to about 15 wt-% nickel and with 0 to about 12 wt-% tungsten. Other metals incorporation methods may be suitable and are contemplated. A suitable density range for the alternative catalyst would be between about 0.60 and about 0.70 g/mL.

The dilute ethylene feed in line 174 may be contacted with the oligomerization catalyst in catalyst bed 178 at a temperature between about 100° and about 400° C. The reaction takes place predominantly in the gas phase at a GHSV 50 to 1000 hr⁻¹ on an ethylene basis. We have found, surprisingly, that despite the presence of impurities in the feed that poison the catalyst and dilution of the ethylene in the feed, that at least about 40 wt-% and as much as about 75 wt-% of the ethylene in the feed stream convert to heavier hydrocarbons. The ethylene will first oligomerize over the catalyst to heavier olefins. Some of the heavier olefins may cyclize over the catalyst, and the presence of hydrogen could facilitate conversion of the olefins to paraffins which are all heavier hydrocarbons than ethylene. However, the cyclized and saturated products are undesirable for cracking to propylene and shall be minimized by proper selection of catalyst and reaction conditions.

The catalyst may remain stable despite the impure feed, but it may be regenerated upon deactivation. Suitable regeneration conditions may include subjecting the catalyst, for example, in situ, to hot air at 500° C. for 3 hours. Activity and selectivity of the regenerated catalyst may be comparable to fresh catalyst.

The oligomerization product stream from the oligomerization reactor in line 180 can be transported to an oligomerization separation unit 182. The oligomerization separation unit may be a simple flash drum to separate a gaseous stream from a liquid stream but can also be more complicated and involve additional incoming stream (not shown), such as an absorber column with unstabilized gasoline or light cycle oil, to increase the recovery efficiency of oligomer. The oligomerization separation unit 182 is in downstream communication with the oligomerization reactor 176. The light raffinate stream in overhead line 184 comprising light gases such as hydrogen, methane, ethane, unreacted olefins and light impurities may be transported to a combustion unit 186 to generate steam in line 187. Alternatively, the light raffinate stream in overhead line 184 may be used in other ways, such as combusted to fire a heater (not shown), and/or to provide a source of flue gas to turn a gas turbine (not shown) to generate power, and/or used for additional hydrogen production in another reforming reactor (not shown). The overhead line 184 is in upstream communication with the combustion unit 186. The liquid bottoms stream in line 185 comprising C₄₊ olefins, and preferably C₄ and C₆ olefins, may be further utilized for producing additional propylene.

In an exemplary embodiment, the light raffinate stream in the overhead line 184 may be passed through a pressure swing adsorption (PSA) unit 194 to remove or recover hydrogen before sending the light raffinate stream in line 184 to the combustion unit 186. The molecules heavier than hydrogen present in the light raffinate stream in line 184 are adsorbed on the adsorbent while hydrogen molecules do not adsorb on the adsorbent and pass through the bed. The adsorbent may be selected from one or more of a silica gel, alumina, activated carbon, molecular sieve including but not limited to zeolite, Metal Organic Framework (MOFs), etc. or their mixture. The PSA 194 may comprise a single layer or multi-layer bed of adsorbent of different composition. The light molecules such as hydrogen leave the PSA 194 at high pressure in line 195 and are recovered. The adsorbed heavy molecules are then desorbed and recovered at a lower pressure from the PSA 194 in a PSA tail gas stream in line 193. The PSA tail gas stream in line 193 may be transported to the combustion unit 186 to generate steam in line 187.

In an embodiment, the PSA 194 may be operated at an adsorption pressure of about 1500 kPa (220 psi) to 3500 kPa (515 psi), and a desorption pressure of about 100 kPa (15 psi) to about 300 kPa (44 psi).

In an alternate embodiment, the light raffinate stream in the overhead line 184 may be passed through a membrane unit 194 to recover or remove hydrogen before sending the light raffinate stream in line 184 to the combustion unit 186. In the membrane unit 194, the light raffinate stream in the overhead line 184 is passed through a membrane at a high pressure. In an exemplary embodiment, the membrane unit 194 may comprise a polyimide or a polyethersulfone blend membrane. Other suitable membranes may include a polymer, porous ceramic, dense ceramic, or metallic membranes. After contact, the molecules heavier than hydrogen present in the light raffinate stream in the overhead line 184 are mostly rejected by the membrane as retentate. The heavy molecules leave the membrane unit 194 at a high pressure and withdrawn in a retentate stream in line 193 from the membrane unit 194. The retentate stream in line 193 may be transported to the combustion unit 186 to generate steam in line 187. Hydrogen present in the light raffinate stream in the overhead line 184 permeates through the membrane at higher selectivity than the retained molecules and the hydrogen is recovered at the other side of membrane at a lower pressure in a hydrogen stream in line 195.

The membrane unit 194 may be operated at a temperature of about 40° C. (104° F.) to about 80° C. (176° F.). The differential pressure across the membrane can be as low as about 70 kPa (10 psi) or as high as 14.5 MPa (2100 psi) depending on many factors such as the particular membrane used, the flow rate of the inlet stream and the availability of a compressor to compress the permeate stream if such compression is desired. In an embodiment, the differential pressure across the membrane may range between about 446 kPa (50 psig) and about 6996 kPa (1000 psig) feed pressure.

Referring back to the oligomerization separation unit 182, the oligomerization liquid bottoms stream taken in line 185, comprising C₄₊ olefins, may be combined with one or more streams for recycle to a second FCC reactor 202 in a second charge line 150 as the second hydrocarbon stream. The second hydrocarbon stream may be preheated to a temperature of about 221° C. (400° F.) to about 704° C. (1300° F.) and charged to the second FCC reactor 202.

The FIGURE shows a second FCC reactor 200 comprising a second reactor unit 202 in which a second hydrocarbon stream in line 150 may be charged through a distributor 216 or more near the base of the second riser 220 and contacted with a second stream of fluid catalyst entering from a regenerated catalyst standpipe 218 and a recirculation catalyst standpipe 219 in a second riser 220. The second hydrocarbon stream may comprise at least 20 wt % olefins, suitably at least 60 wt % olefins and preferably at least 70 wt % olefins. The second hydrocarbon stream may comprise at least 1 wt % paraffins, suitably at least 15 wt % paraffins and preferably at least 25 wt % paraffins. The second hydrocarbon stream may comprise once cracked C4 to C7 hydrocarbons. The second riser 220 and the second reactor unit 202 may be in upstream and downstream communication with the first riser 20 and the secondary absorber 118.

The contacting may occur in a narrow second riser 220, extending upwardly to the bottom of a second reactor vessel 222. The contacting of the second hydrocarbon feedstock and the second stream of fluid catalyst is fluidized by gas such as steam from a fluidizing distributor 224. In an embodiment, heat from the catalyst vaporizes the second hydrocarbon feedstock, and the second hydrocarbon feedstock is thereafter cracked to a second cracked product stream of lighter molecular weight in the presence of the second catalyst stream as both are transferred up the riser 220 into the second reactor vessel 222 providing a second mixture of catalyst and product gases.

The pressure in the second riser 220 may be about 200 kPa (29 psia) to about 450 kPa (65 psia), but it could be lower. A steam rate of about 3 to about 7 wt % of the second hydrocarbon feedstock is added to the second riser 220. Inevitable side reactions occur in the second riser 220 leaving coke deposits on the catalyst that lower catalyst activity to provide a spent catalyst stream. The second cracked product stream in the second mixture of catalyst and product gases is thereafter separated from the spent catalyst stream using cyclonic separators which may include one or two stages of cyclones 262 in the second reactor vessel 222. A gaseous, second cracked product stream exits the second reactor vessel 222 through a second product outlet 231 to line 232 for transport to the downstream product recovery section 90.

The second stream of fluid catalyst can compromise less than about 20 wt %, preferably less than about 5 wt % of the first component and at least 20% by weight, of the second component. In one preferred embodiment, the second stream of fluid catalyst can include at least about 20 wt % of a ZSM-5 zeolite and less than about 20 wt %, preferably less than about 5 wt % of a Y-zeolite. In another preferred embodiment, the second stream of fluid catalyst can predominantly comprise the second component and in a further embodiment can contain only the second component, preferably a ZSM-5 zeolite, as the catalyst.

Process conditions in the second riser 212 will be more severe than in the first riser 20 because the second hydrocarbon stream has lower average molecular weight and higher olefin content, making it more selective towards cracking to propylene under more severe conditions than the first hydrocarbons stream, yet producing lower level of byproduct such as coke. Olefins in the second hydrocarbon stream that could not directly crack to form propylene, such as C4 olefins, can first go through oligomerization and convert to suitable heavier olefins, then crack back to propylene in the second riser under the process conditions. The second riser 220 may operate at one or more of the following conditions relative to the first riser 20: a higher outlet temperature, a lower hydrocarbon partial pressure or a different catalyst density. Hydrocarbon partial pressure is reduced by reducing the total pressure in the second riser 220 independent of the pressure in the first FCC reactor 10 and perhaps adjusting the steam rate to the second riser 212.

Conditions in the second riser 220 may include a cracking reaction temperature of 400° to 650° C., preferably about 565° C. to about 635° C. at the reactor outlet. The cracking occurs at an absolute pressure between about 100 kPa (14 psia) to about 506 kPa (74 psia), preferably between about 138 kPa (20 psia) to about 310 kPa (45 psia). A steam flow rate of about 5 to about 25 wt % of the second hydrocarbon stream is added to the second riser 220.

The spent or coked catalyst requires regeneration for further use. The FIGURE shows a single regenerator 14, but use of two separate regenerators is also contemplated. The spent catalyst stream, after separation from the second cracked product stream by means of a disengagement device 254 in a second disengagement chamber 256, falls into a stripping section 234 where steam is injected through a distributor 235 to purge any residual hydrocarbon vapor. A transport conduit 260 carries the hydrocarbon vapors, including stripped hydrocarbons, stripping media and entrained catalyst to one or two stages of cyclones 262 in the second reactor vessel 222 which separates coked catalyst from the hydrocarbon vapor stream. After the stripping operation, the stripped coked catalyst is carried to the catalyst regenerator 14 through a spent catalyst standpipe 236. Another portion of the stripped coked catalyst may be recycled to the second riser 220 by the recirculation catalyst standpipe 219 without undergoing regeneration.

All or a portion of the liquid bottoms stream in line 185 comprising C₄₊ olefins, and preferably C₄ and C₆ olefins, may be fed to a catalytic reaction unit 190 via line 189 controlled by a valve thereon. The catalytic reaction unit 190 may receive the second C₄ hydrocarbon stream via line 179, controlled by a valve thereon. Additional feeds to a catalytic reaction unit 190 may be fed via line 191. The catalytic reaction unit 190 may catalytically convert feedstream(s) to a converted product stream in line 196. The converted product stream in line 196 may be recycled to a second FCC reactor 202 via line 198 in a second charge line 150 as at least a portion of the second hydrocarbon stream. All or a portion of the converted product stream may be further processed by routing to the product recovery section 90 via line 197, controlled by a valve thereon.

In an aspect, a catalytic reaction unit 190 may comprise a cracking process. An exemplary cracking process is the olefin cracking process (OCP), available from UOP LLC. A catalyst used within a cracking process in catalytic reaction unit 190 may comprise an MFI zeolite. A MFI zeolite may have a silica-to-alumina ratio of between approximately 400 and approximately 1200, or between about 600 and 1100. A catalyst may further comprise a binder, which may include alumina. A catalytic reaction temperature in a catalytic reaction unit 190 may be between about 500° C. and about 650° C. A catalytic reaction pressure may be between about 1 and 20 psig, and a catalytic reaction unit may operate at a WHSV of between about 1 and 20 II′ or between about 5 and 15 II′.

In an aspect, a catalytic reaction unit 190 may comprise an oligomerization process. An exemplary oligomerization process is the Catolene process available from UOP LLC. A catalyst used within a cracking process in catalytic reaction unit 190 may comprise solid phosphoric acid (SPA). The SPA catalyst refers to a solid catalyst that contains as a principal ingredient an acid of phosphorous such as ortho-, pyro- or tetra-phosphoric acid. SPA catalyst is normally formed by mixing the acid of phosphorous with a siliceous solid carrier to form a wet paste. This paste may be calcined and then crushed to yield catalyst particles or the paste may be extruded or pelleted prior to calcining to produce more uniform catalyst particles. The carrier is preferably a naturally occurring porous silica containing material such as kieselguhr, kaolin, infusorial earth and diatomaceous earth. A minor amount of various additives such as mineral talc, fuller's earth and iron compounds including iron oxide may be added to the carrier to increase its strength and hardness. The combination of the carrier and the additives preferably comprises about 15-30 wt % of the catalyst, with the remainder being the phosphoric acid. The additive may comprise about 3-20 wt % of the total carrier material. Variations from this composition such as a lower phosphoric acid content are possible. Further details as to the composition and production of SPA catalysts may be obtained from U.S. Pat. Nos. 3,050,472, 3,050,473 and 3,132,109 and from other references. A catalytic reaction temperature in a catalytic reaction unit 190 may be between about 100° C. and about 300° C. or between about 115° C. and about 225° C. A catalytic reaction pressure may be between about 100 psig and 1000 psig or between about 400 psig and about 700 psig, and a catalytic reaction unit may operate at a LHSV of between about 0.5 and 5 h⁻¹ or between about 1 and 2 h⁻¹.

In an aspect, a catalytic reaction unit 190 may comprise a metathesis process. Metathesis processes convert two olefins to a third olefin. A catalyst used within a metathesis process in catalytic reaction unit 190 may comprise tungsten impregnated on a silica support. Pretreatment of a metathesis catalyst may be necessary prior to reaction. Pretreatment conditions may involve reduction in hydrogen at a temperature of about 500° C. followed by a treatment with ethylene at a temperature of about 250° C. Ethylene may be fed via alternate feed line 191. Feed streams rich in butenes such as the second C₄ hydrocarbon stream in line 147 are preferred when a catalytic reaction unit 190 comprises metathesis. A catalytic reaction temperature in a catalytic reaction unit 190 may be between about 300° C. and about 400° C. A catalytic reaction pressure may be between about 300 and 700 psig, and a catalytic reaction unit may operate at a LHSV of between about 0.1 and 10 h⁻¹ or between about 0.5 and 3 h⁻¹.

### EXAMPLES

The following examples are provided to illustrate one or more preferred embodiments of the present disclosure but are not limited embodiments thereof. Numerous variations can be made to the following examples that lie within the scope of the present disclosure.

### Example 1

A nickel on amorphous silica-alumina extrudate catalyst was prepared through the following steps:

A silica-alumina base was formed by extruding a mixture dough through 1.59 mm openings in a cylindrical die plate and broken into pieces prior to calcination at 550° C. The dough was prepared by combining an amorphous silica-alumina having a silica-to alumina ratio of about 2.6 provided by CCIC, and pseudo boehmite provided under the Catapal trademark in a weight ratio of 85:15. The pseudo-boehmite was peptized with nitric acid before mixture with the amorphous silica-alumina. A surfactant provided under the Antarox trademark and water in sufficient quantity to wet the dough were also added to the mixture.

The nickel was then introduced to the base by dissolving 3.37 grams Ni(NO₃)₂·6H₂O in 32.08 grams of deionized water and adding the nickel solutions in fourths to the amorphous silica-alumina base and shaking vigorously between additions. The impregnated base was then dried at 110° C. for 3 hours, then calcined at 500° C. for 3 hours to convert the nickel to oxide form. The final catalyst A contains 1.63 wt % nickel.

### Example 2

A nickel on amorphous silica-alumina oil-dropped spherical base catalyst was synthesized via the procedures given hereinabove for an alternative catalyst. After forming the amorphous silica-alumina oil-dropped spherical base at average diameters of 3.175 mm and a silica-to-alumina ratio of about 3, the nickel was introduced to the base following the same method described in Example 1, except that the solution concentration and volume adjusted to match the oil-dropped spherical base and achieving metal level of 0.44 wt-% nickel on the final catalyst B.

### Example 3

A sodium modified nickel on amorphous silica-alumina extrudate catalyst was prepared from the same calcined silica-alumina extrudate described in Example 1, but using excess solution impregnation method for metal loading:

A nickel solution was prepared by dissolving 2.98 grams Ni(NO₃)₂·6H₂O in 124 grams deionized water. The calcined extruded base from Example 1 contacted the nickel solution in an evaporation flask and the solution-base mixture was rolled at 50° C. for 2 hours, and then at 100° C. for another 2 hours to vaporize the water.

The nickel impregnated base was then further dried at 100° C. for 3 hours and calcined at 500° C. for 4 hours before sodium impregnation, which followed the same procedure as nickel impregnation, except the solution was made by dissolving 6.84 grams NaNO₃ in 131 grams deionized water. The sodium and nickel impregnated base was again dried at 100° C. for 3 hours and calcined at 500° C. for 4 hours. The final catalyst C contained 1 wt % Ni and 3 wt % Na.

### Example 4

Catalyst A was tested for olefin oligomerization at 300° C., 1000 psig and 409 OGHSV (olefin gas hourly space velocity) in a fixed bed operation over 10 ml of catalyst. The feed consisted of 30.6 wt % ethylene, 20.0 wt % ethane, 26.2 wt % methane, 1.25 wt % hydrogen, 17.5 wt % nitrogen, 1.3 wt % carbon monoxide, 3.2 wt % carbon dioxide, 17.5 wppm hydrogen sulfide, 10 wppm ammonia and saturate level of moisture. The online GC analysis was not able to differentiate olefin vs. paraffin selectivity for the C9+ products because they were too heavy and which made up most of the product using Catalyst A.

Catalyst B was tested for olefin oligomerization at 299° C., 900 psig and 202 OGHSV in a fixed bed operation over 27.5 ml of catalyst. The feed consisted of 46.2 wt % ethylene, 22.9 wt % ethane, 25.4 wt % methane, 3.0 wt % hydrogen, 1.0 wt % carbon monoxide, 1.5 wt % carbon dioxide, 37 wppm hydrogen sulfide and 108 wppm of acetylene.

Catalyst C was tested for olefin oligomerization at 285° C., 900 psig and 173 OGHSV in a fixed bed operation over 32.0 ml of catalyst. The feed was the same as the test of Catalyst B above. Results of these tests are listed in Table 1.

TABLE 1
Olefin Oligomerization Product Distribution

| | Selectivity | | | C4 to C8 Olefin wt % in C₄⁺ |
|---|---|---|---|---|
| Catalyst | C₂-C₃ | C₄-C₈ | C₉⁺ | Total Product |
| A | 1.4 | 0.4 | 98.2 | — |
| B | 3.4 | 82.4 | 14.2 | 57.6 |
| C | 26.9 | 72.2 | 0.9 | 90.7 |

The results showed that suitable combination of catalyst and reaction condition could increase C₄-C₈ hydrocarbons product selectivity as well as the product olefin level of ethylene oligomerization reaction in the presence of contaminants. A higher level of C₄-C₈ olefin product is expected to increase the production of propylene when the oligomer is sent to fluidized catalytic cracking.

### Specific Embodiments

While the following is described in conjunction with specific embodiments, it will be understood that this description is intended to illustrate and not limit the scope of the preceding description and the appended claims.

A first embodiment of the present disclosure is a process for production of propylene comprising contacting a hydrocarbon feed stream with a fluidized catalyst to produce a cracked product stream; separating the cracked product stream in a main column; separating an overhead stream from the main column into a dilute ethylene stream; contacting the dilute ethylene stream with an olefin oligomerization catalyst; converting ethylene in the dilute ethylene stream to an oligomerized product stream; and contacting at least a portion of the oligomerized product stream with a fluidized catalyst to convert at least a portion of the oligomerized product stream to propylene. An embodiment of the present disclosure is one, any or all of prior embodiments in this paragraph up through the first embodiment in this paragraph further comprising separating the oligomerized product stream to obtain a C4+ olefin stream and a light raffinate stream and contacting the C4+ olefin stream with fluidized catalyst to convert at least a portion of the C4+ olefins to propylene. An embodiment of the present disclosure is one, any or all of prior embodiments in this paragraph up through the first embodiment in this paragraph wherein the step of contacting the hydrocarbon feed stream with a fluidized catalyst is carried out in a first riser at first process conditions and the step of contacting the C4+ olefin stream with a fluidized catalyst is carried out in a second riser at second process conditions. An embodiment of the present disclosure is one, any or all of prior embodiments in this paragraph up through the first embodiment in this paragraph wherein the second process conditions are more severe than the first process conditions. An embodiment of the present disclosure is one, any or all of prior embodiments in this paragraph up through the first embodiment in this paragraph wherein generation of the cracked product stream produces a spent catalyst stream. An embodiment of the present disclosure is one, any or all of prior embodiments in this paragraph up through the first embodiment in this paragraph further comprising regenerating the stream of spent catalyst by combustion of coke from the spent catalyst to provide regenerated catalyst. An embodiment of the present disclosure is one, any or all of prior embodiments in this paragraph up through the first embodiment in this paragraph wherein the combustion step occurs at a temperature between about 670 and about 750° C. An embodiment of the present disclosure is one, any or all of prior embodiments in this paragraph up through the first embodiment in this paragraph wherein the olefin oligomerization catalyst comprises an amorphous silica-alumina base and a metal or metals selected from the group consisting of Group 6, 8, 9 and 10 in the periodic table. An embodiment of the present disclosure is one, any or all of prior embodiments in this paragraph up through the first embodiment in this paragraph wherein the olefin oligomerization catalyst further comprises a metal or metals selected from the group consisting of Group 1 and 2 in the periodic table. An embodiment of the present disclosure is one, any or all of prior embodiments in this paragraph up through the first embodiment in this paragraph wherein ethylene in the dilute ethylene stream is converted with at least 60 wt-% selectivity to C4-C8 hydrocarbons. An embodiment of the present disclosure is one, any or all of prior embodiments in this paragraph up through the first embodiment in this paragraph further comprising separating an overhead stream from the main column into a second hydrocarbon stream. An embodiment of the present disclosure is one, any or all of prior embodiments in this paragraph up through the first embodiment in this paragraph further comprising contacting the light raffinate stream with an adsorbent in a pressure swing adsorption (PSA) unit to remove hydrogen from the light raffinate stream and provide a PSA tail gas stream comprising ethylene and contacting the PSA tail gas stream with the olefin oligomerization catalyst. An embodiment of the present disclosure is one, any or all of prior embodiments in this paragraph up through the first embodiment in this paragraph further comprising contacting the dilute ethylene stream with an adsorbent in a pressure swing adsorption (PSA) unit to recover hydrogen from the dilute ethylene stream and provide a PSA tail gas stream comprising unreacted olefins.

A second embodiment of the present disclosure is a process for production of propylene comprising contacting a hydrocarbon feed stream with a fluidized catalyst to produce a cracked product stream; separating the cracked product stream in a main column; separating an overhead stream from the main column into a dilute ethylene stream; separating an overhead stream from the main column into a second hydrocarbon stream comprising C4 olefins; contacting the dilute ethylene stream with an olefin oligomerization catalyst; converting ethylene in the dilute ethylene stream to an oligomerized product stream; separating the oligomerized product stream to obtain a C4+ olefin stream and a light raffinate stream; reacting at least a portion of the C4+ olefin stream in a catalytic reaction unit to provide a converted product stream; and contacting at least a portion of the converted product stream with a fluidized catalyst to convert at least a portion of the C4+ olefin to propylene. An embodiment of the present disclosure is one, any or all of prior embodiments in this paragraph up through the second embodiment in this paragraph wherein the step of contacting the hydrocarbon feed stream with a fluidized catalyst is carried out in a first riser at first process conditions. An embodiment of the present disclosure is one, any or all of prior embodiments in this paragraph up through the second embodiment in this paragraph wherein the step of contacting the converted product stream with a fluidized catalyst is carried out in a second riser at second process conditions. An embodiment of the present disclosure is one, any or all of prior embodiments in this paragraph up through the first embodiment in this paragraph wherein the second process conditions are more severe than the first process conditions. An embodiment of the present disclosure is one, any or all of prior embodiments in this paragraph up through the second embodiment in this paragraph wherein generation of the cracked product stream produces a spent catalyst stream. An embodiment of the present disclosure is one, any or all of prior embodiments in this paragraph up through the second embodiment in this paragraph further comprising regenerating the stream of spent catalyst by combustion of coke from the spent catalyst to provide regenerated catalyst. An embodiment of the present disclosure is one, any or all of prior embodiments in this paragraph up through the second embodiment in this paragraph wherein the combustion step occurs at a temperature between about 670 and about 750° C. An embodiment of the present disclosure is one, any or all of prior embodiments in this paragraph up through the second embodiment in this paragraph wherein the olefin oligomerization catalyst comprises an amorphous silica-alumina base and a metal or metals selected from the group consisting of Group 6, 8, 9 and 10 in the periodic table. An embodiment of the present disclosure is one, any or all of prior embodiments in this paragraph up through the second embodiment in this paragraph wherein ethylene in the dilute ethylene stream is converted with at least 60 wt-% selectivity to C4-C8 hydrocarbons. An embodiment of the present disclosure is one, any or all of prior embodiments in this paragraph up through the second embodiment in this paragraph wherein the catalytic reaction unit comprises a cracking process. An embodiment of the present disclosure is one, any or all of prior embodiments in this paragraph up through the second embodiment in this paragraph wherein the catalytic reaction unit comprises oligomerization. An embodiment of the present disclosure is one, any or all of prior embodiments in this paragraph up through the second embodiment in this paragraph wherein the catalytic reaction unit comprises metathesis, and at least a portion of the converted product stream is further processed to recover a propylene stream. An embodiment of the present disclosure is one, any or all of prior embodiments in this paragraph up through the second embodiment in this paragraph further comprising contacting the second hydrocarbon stream with the olefin oligomerization catalyst. An embodiment of the present disclosure is one, any or all of prior embodiments in this paragraph up through the second embodiment in this paragraph wherein the dilute ethylene stream comprises between about 10 and about 65 wt-% ethylene, between about 20 and about 55 wt-% methane, and at least one impurity selected from the group consisting of at least about 0.1 wt-% carbon monoxide, at least about 1 wppm hydrogen sulfide, at least about 1 wppm ammonia, at least about 5 wt-% hydrogen and at least about 0.1 wt-% carbon dioxide.

A third embodiment of the present disclosure is a process for production of propylene comprising contacting a hydrocarbon feed stream with a fluidized catalyst to produce a cracked product stream; separating the cracked product stream in a main column; separating an overhead stream from the main column into a dilute ethylene stream comprising between about 10 and about 65 wt-% ethylene, between about 20 and about 55 wt-% methane, and at least one impurity selected from the group consisting of at least about 0.1 wt-% carbon monoxide, at least about 1 wppm hydrogen sulfide, at least about 1 wppm ammonia, at least about 0.5 wt-% hydrogen and at least about 0.1 wt-% carbon dioxide; separating an overhead stream from the main column into a second hydrocarbon stream comprising C4 olefins; contacting the dilute ethylene stream and the second hydrocarbon stream with an olefin oligomerization catalyst; converting ethylene in the dilute ethylene stream to an oligomerized product stream; separating the oligomerized product stream to obtain a C4+ olefin stream and a light raffinate stream; reacting at least a portion of the C4+ olefin stream in a catalytic reaction unit to provide a converted product stream; and contacting at least a portion of the converted product stream with a fluidized catalyst to convert at least a portion of the C4+ olefin to propylene. An embodiment of the present disclosure is one, any or all of prior embodiments in this paragraph up through the third embodiment in this paragraph wherein the step of contacting the hydrocarbon feed stream with a fluidized catalyst is carried out in a first riser at first process conditions. An embodiment of the present disclosure is one, any or all of prior embodiments in this paragraph up through the third embodiment in this paragraph wherein the step of contacting the converted product stream with a fluidized catalyst is carried out in a second riser at second process conditions. An embodiment of the present disclosure is one, any or all of prior embodiments in this paragraph up through the first embodiment in this paragraph wherein the second process conditions are more severe than the first process conditions. An embodiment of the present disclosure is one, any or all of prior embodiments in this paragraph up through the third embodiment in this paragraph wherein generation of the cracked product stream produces a spent catalyst stream. An embodiment of the present disclosure is one, any or all of prior embodiments in this paragraph up through the third embodiment in this paragraph further comprising regenerating the stream of spent catalyst by combustion of coke from the spent catalyst to provide regenerated catalyst. An embodiment of the present disclosure is one, any or all of prior embodiments in this paragraph up through the third embodiment in this paragraph wherein the combustion step occurs at a temperature between about 670 and about 750° C. An embodiment of the present disclosure is one, any or all of prior embodiments in this paragraph up through the third embodiment in this paragraph wherein the olefin oligomerization catalyst comprises an amorphous silica-alumina base and a metal or metals selected from the group consisting of Group 6, 8, 9 and 10 in the periodic table. An embodiment of the present disclosure is one, any or all of prior embodiments in this paragraph up through the third embodiment in this paragraph wherein ethylene in the dilute ethylene stream is converted with at least 60 wt-% selectivity to C4-C8 hydrocarbons. An embodiment of the present disclosure is one, any or all of prior embodiments in this paragraph up through the third embodiment in this paragraph wherein the catalytic reaction unit comprises a cracking process. An embodiment of the present disclosure is one, any or all of prior embodiments in this paragraph up through the third embodiment in this paragraph wherein the catalytic reaction unit comprises oligomerization. An embodiment of the present disclosure is one, any or all of prior embodiments in this paragraph up through the third embodiment in this paragraph wherein the catalytic reaction unit comprises metathesis, and at least a portion of the converted product stream is further processed to recover a propylene stream.

A fourth embodiment of the present disclosure is a process for production of propylene comprising contacting a hydrocarbon feed stream with a fluidized catalyst in a first riser at first process conditions to produce a first cracked product stream and a spent catalyst stream; separating the cracked product stream in a main column; separating an overhead stream from the main column into a dilute ethylene stream comprising between about 10 and about 65 wt-% ethylene, between about 20 and about 55 wt-% methane, and at least one impurity selected from the group consisting of at least about 0.1 wt-% carbon monoxide, at least about 1 wppm hydrogen sulfide, at least about 1 wppm ammonia, at least about 0.5 wt-% hydrogen and at least about 0.1 wt-% carbon dioxide; separating an overhead stream from the main column into a second hydrocarbon stream comprising C4 olefins; contacting the dilute ethylene stream and the second hydrocarbon stream with an olefin oligomerization catalyst; converting ethylene in the dilute ethylene stream to an oligomerized product stream; separating the oligomerized product stream to obtain a C4+ olefin stream and a light raffinate stream; reacting at least a portion of the C4+ olefin stream in a catalytic reaction unit to provide a converted product stream; and contacting at least a portion of the converted product stream with a fluidized catalyst in a second riser at second process conditions to convert at least a portion of the C4+ olefin to propylene, wherein the second process conditions are more severe than the first process conditions.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for production of propylene comprising:
contacting a hydrocarbon feed stream with a fluidized catalyst to produce a cracked product stream;
separating said cracked product stream in a main column;
separating an overhead stream from the main column into a dilute ethylene stream;
contacting the dilute ethylene stream with an olefin oligomerization catalyst;
converting ethylene in the dilute ethylene stream to an oligomerized product stream; and
contacting at least a portion of the oligomerized product stream with a fluidized catalyst to convert at least a portion of the oligomerized product stream to propylene.

2. The process of claim 1 further comprising separating the oligomerized product stream to obtain a C₄⁺ olefin stream and a light raffinate stream and contacting the C₄⁺ olefin stream with fluidized catalyst to convert at least a portion of the C₄⁺ olefins to propylene.

3. The process of claim 1 wherein the step of contacting the hydrocarbon feed stream with a fluidized catalyst is carried out in a first riser at first process conditions and the step of contacting the C₄⁺ olefin stream with a fluidized catalyst is carried out in a second riser at second process conditions.

4. The process of claim 3 wherein the second process conditions are more severe than the first process conditions.

5. The process of claim 1 wherein the olefin oligomerization catalyst comprises an amorphous silica-alumina base and a metal or metals selected from the group consisting of Group 6, 8, 9 and 10 in the periodic table.

6. The process of claim 5 wherein the olefin oligomerization catalyst further comprises a metal or metals selected from the group consisting of Group 1 and 2 in the periodic table.

7. The process of claim 1 wherein ethylene in the dilute ethylene stream is converted with at least 60 wt-% selectivity to C4-C8 hydrocarbons.

8. The process of claim 1 further comprising separating an overhead stream from the main column into a second hydrocarbon stream.

9. The process of claim 2 further comprising:
contacting said light raffinate stream with an adsorbent in a pressure swing adsorption (PSA) unit to remove hydrogen from said light raffinate stream and provide a PSA tail gas stream comprising ethylene; and
contacting said PSA tail gas stream with the olefin oligomerization catalyst.

10. The process of claim 1 further comprising contacting said dilute ethylene stream with an adsorbent in a pressure swing adsorption (PSA) unit to recover hydrogen from said dilute ethylene stream and provide a PSA tail gas stream comprising unreacted olefins.

11. A process for production of propylene comprising:
contacting a hydrocarbon feed stream with a fluidized catalyst to produce a cracked product stream;
separating said cracked product stream in a main column;
separating an overhead stream from the main column into a dilute ethylene stream;
separating an overhead stream from the main column into a second hydrocarbon stream comprising C₄ olefins;
contacting the dilute ethylene stream with an olefin oligomerization catalyst;
converting ethylene in the dilute ethylene stream to an oligomerized product stream;
separating the oligomerized product stream to obtain a C₄⁺ olefin stream and a light raffinate stream;
reacting at least a portion of the C₄⁺ olefin stream in a catalytic reaction unit to provide a converted product stream; and
contacting at least a portion of the converted product stream with a fluidized catalyst to convert at least a portion of the C₄⁺ olefin to propylene.

12. The process of claim 11 wherein the step of contacting the hydrocarbon feed stream with a fluidized catalyst is carried out in a first riser at first process conditions.

13. The process of claim 11 wherein the step of contacting the converted product stream with a fluidized catalyst is carried out in a second riser at second process conditions.

14. The process of claim 13 wherein the second process conditions are more severe than the first process conditions.

15. The process of claim 14 wherein the olefin oligomerization catalyst comprises an amorphous silica-alumina base and a metal or metals selected from the group consisting of Group 6, 8, 9 and 10 in the periodic table.

16. The process of claim 14 wherein ethylene in the dilute ethylene stream is converted with at least 60 wt-% selectivity to C₄-C₈ hydrocarbons.

17. The process of claim 14 wherein the catalytic reaction unit comprises metathesis, and at least a portion of the converted product stream is further processed to recover a propylene stream.

18. The process of claim 14 wherein the catalytic reaction unit comprises oligomerization.

19. The process of claim 14 further comprising contacting the second hydrocarbon stream with the olefin oligomerization catalyst.

20. The process of claim 14 wherein the dilute ethylene stream comprises between about 10 and about 65 wt-% ethylene, between about 20 and about 55 wt-% methane, and at least one impurity selected from the group consisting of at least about 0.1 wt-% carbon monoxide, at least about 1 wppm hydrogen sulfide, at least about 1 wppm ammonia, at least about 0.5 wt-% hydrogen and at least about 0.1 wt-% carbon dioxide.

21. The process of claim 11 wherein the catalytic reaction unit comprises a cracking process.

22. A process for production of propylene comprising:
contacting a hydrocarbon feed stream with a fluidized catalyst in a first riser at first process conditions to produce a first cracked product stream and a spent catalyst stream;
separating said cracked product stream in a main column;
separating an overhead stream from the main column into a dilute ethylene stream comprising between about 10 and about 65 wt-% ethylene, between about 20 and about 55 wt-% methane, and at least one impurity selected from the group consisting of at least about 0.1 wt-% carbon monoxide, at least about 1 wppm hydrogen sulfide, at least about 1 wppm ammonia, at least about 0.5 wt-% hydrogen and at least about 0.1 wt-% carbon dioxide;
separating an overhead stream from the main column into a second hydrocarbon stream comprising C₄ olefins;
contacting the dilute ethylene stream and the second hydrocarbon stream with an olefin oligomerization catalyst;
converting ethylene in the dilute ethylene stream to an oligomerized product stream;
separating the oligomerized product stream to obtain a C₄⁺ olefin stream and a light raffinate stream;
reacting at least a portion of the C₄⁺ olefin stream in a catalytic reaction unit to provide a converted product stream; and
contacting at least a portion of the converted product stream with a fluidized catalyst in a second riser at second process conditions to convert at least a portion of the C₄⁺ olefin to propylene, wherein the second process conditions are more severe than the first process conditions.
